(19) United States
(12) Patent Application Publication
(10) Pub. No.: US 2019/0000866 A1
(43) Pub. Date: Jan. 3, 2019

(54) FOOD BASED DELIVERY OF THERAPEUTIC AGENT FOR TREATMENT OF HEPATIC ENCEPHALOPATHY

(71) Applicant: ColonaryConcepts LLC, Palm Beach Gardens, FL (US)

(72) Inventors: Corey A. SIEGEL, Palm Beach Gardens, FL (US); Joshua KORZENIK, Palm Beach Gardens, FL (US); Michael K. ALLIO, Palm Beach Gardens, FL (US); Herbert B. STERN, Palm Beach Gardens, FL (US)

(21) Appl. No.: 16/067,759

(22) PCT Filed: Jan. 6, 2017
(86) PCT No.: PCT/US2017/012620

§ 371 Date: Jul. 2, 2018

Related U.S. Application Data
(60) Provisional application No. 62/276,685, filed on Jan. 8, 2016.
(60) Provisional application No. 62/276,683, filed on Jan. 8, 2016.

(51) Int. Cl. A61K 31/7036; A61K 31/075; A61K 31/7016; A61K 31/437; A61K 31/4164; A61K 31/426; A61K 31/192; A61K 31/198; A61K 9/00; A61K 47/44; A23L 33/125; A23L 2/52; A23L 33/115

(52) U.S. Cl. CPC A61K 31/7036; A23V 2002/00; A61K 31/7016; A61K 31/437; A61K 31/4164; A61K 31/426; A61K 31/192; A61K 31/198; A61K 9/0053; A61K 9/0095; A61K 47/44; A23L 33/125; A23L 2/52; A23L 33/115; A61K 31/075

(57) ABSTRACT

Described herein are compositions, methods, and kits for treating hepatic encephalopathy. A composition for treating or preventing hepatic encephalopathy comprises one or more therapeutic agents and a delivery modality comprising an edible food, beverage, candy, or gum. The one or more therapeutic agents are mixed with one or more food ingredients so that the therapeutic agent is incorporated in the delivery modality.

### CROSS-REFERENCE

This application claims the benefit of U.S. Provisional Application No. 62/276,683, filed Jan. 8, 2016 and U.S. Provisional Application No. 62/276,685, filed Jan. 8, 2016, which applications are both incorporated herein by reference.

### BACKGROUND

Hepatic encephalopathy is the occurrence of mental confusion, altered level of consciousness, and ultimately coma due to liver failure. The liver normally functions to remove toxins from the blood stream, however, when the liver fails it is unable to do so and the brain is thereby exposed to neurotoxic compositions in the blood stream which leads to mental confusion, altered level of consciousness, and ultimately coma.

A main source of toxins filtered by a healthy liver is the GI tract, which produces toxins that are byproducts of ingested foods, other ingested substances, and the flora of the GI tract. As such, a major treatment approach in liver failure is to reduce the toxin production by or toxin absorption from the GI tract in order to compensate for the decreased ability of the liver to remove the toxins produced by the GI tract from the blood stream.

### SUMMARY

Described herein are compositions, methods, and kits for providing therapy to an individual to treat and/or prevent hepatic encephalopathy. In general, the compositions, methods, and kits described herein provide a delivery vehicle for effective delivery of therapeutic agents to an individual suffering from liver disease.

The compositions, methods, and kits described herein are configured to inter alia promote compliance with a treatment regimen provided to an individual with hepatic failure and hepatic encephalopathy.

Hepatic encephalopathy is associated with impairment of normal cognitive function due to confusion and memory loss associated with hepatic encephalopathy, and this impairment of cognitive function makes it extremely difficult for individuals with hepatic encephalopathy to carry out repeated complex tasks such as preparing and timely taking their therapeutic agent regimen. Typically a plurality of therapeutic agents are prescribed for the treatment of both hepatic failure as well as hepatic encephalopathy which are taken at different times, and some of which are in powder form and need to be measured out by the individual taking the therapy and mixed into solution. Because of the relative complexity of the traditional treatment regimen for hepatic encephalopathy, it is extremely difficult for individuals suffering hepatic encephalopathy to carry out the various tasks required of compliance including preparing the therapeutic agents (i.e. mixing solutions) and remembering to take the therapeutic agents. As such, typically, compliance of hepatic encephalopathy patients with therapeutic regiment is relatively poor. In addition, lactulose, a traditional therapeutic is unpalatable to most individuals and as such there is an additional factor (i.e. poor palatability) that tends to lead to poor compliance.

Compliance with the treatment regimen provided to an individual with hepatic failure and hepatic encephalopathy is promoted by the compositions, methods, and kits described herein in at least three ways: 1) The delivery vehicle provides a complete pre-measured dosage of all therapeutics, 2) The delivery vehicle provides a formulation that in some embodiments is configured to contain all of the therapeutics needed for treatment and/or prevention of hepatic encephalopathy within a single formulation, and 3) The delivery vehicle, in some embodiments, is configured to mask an unpalatable flavor associated with one or more therapeutic agents.

Delivery of a Pre-Measured Dose

Typically, the traditional therapeutic regimen for the treatment and prevention of hepatic encephalopathy includes lactulose, a laxative agent. A laxative, such as lactulose, is provided to patients with hepatic failure in an effort to clear the colon and thus reduce the absorption of gastrointestinal contents that become toxins when absorbed into the blood stream of a patient in hepatic failure, because these patients are unable to filter these gastrointestinal contents from the blood stream due to the failure of the malfunctioning liver to do so. Lactulose is typically provided to an individual as a powder that the individual must measure out and mix in an appropriate amount of liquid. Measuring the dosage of a powder and mixing it into a solution is a relatively complex task that is difficult for those with cognitive impairment. In addition, typically, the treatment of hepatic encephalopathy requires multiple daily doses of laxative, so individuals are required to carry out the relatively complex mixing task more than once a day. As such, in the cognitively impaired hepatic encephalopathy patients, compliance with the laxative in the treatment regimen is poor.

Described herein are compositions, methods, and kits wherein the laxative that is provided to an individual with hepatic encephalopathy is provided within a delivery vehicle in a pre-measured quantity. In some embodiments of the compositions, methods, and kits described herein, one or more laxatives are mixed together with one or more food ingredients so that the laxative provided to an individual with hepatic encephalopathy is in a pre-measured and pre-mixed formulation. In this way compliance of individuals with hepatic encephalopathy is improved, as they are no longer required to carry out the relatively complex mixing step for preparing the laxative portion of their therapy, because the laxative is already incorporated into an edible food item in the proper amount.

Delivery of a Complete Therapeutic Regimen

Typically, a traditional therapeutic regimen for the treatment and prevention of hepatic encephalopathy includes a plurality of therapeutic agents including laxatives, antibiotics, and in some instances additionally antivirals as well. Many of these different therapeutics must typically be taken by an individual with hepatic encephalopathy more than once a day, and in some instances different therapeutics must be taken at different times. This relatively complex therapeutic schedule is difficult for the cognitively impaired hepatic encephalopathy patient, and as such leads to poor compliance with the regimen.

Described herein are compositions, methods, and kits that provide a delivery vehicle that contains a complete therapeutic regimen. That is, in some embodiments of the compositions described herein, the composition provides a formulation that contains all of the therapeutic agents combined. For example, in some embodiments of the compositions described herein, a composition comprises a laxative and one or more antibiotics mixed together with one or more food ingredients to form an edible food bar. For example, in some embodiments of the kits described herein, a kit comprises one or more food items that in total contain the entire therapeutic regimen provided to an individual to treat or prevent hepatic encephalopathy. In such embodiments, for example, kits may comprise different food items corresponding to different meals in the day. In this way, if different therapeutic agents are to be provided at different times of the day (i.e. not together at the same time), then, for example, a first therapeutic agent may be included in a breakfast bar and a pasta based lunch dish may include the first therapeutic agent together with a second therapeutic agent. In this way, compliance of individuals with hepatic encephalopathy is improved as an edible food item or meal containing kit provides a formulation containing the therapeutic agents that they need to take for their therapeutic regimen, thus avoiding the relatively complex task for a cognitively impaired patient of remembering to take a plurality of therapeutic agents more than once a day.

Described herein are compositions, methods, and kits wherein at least one laxative that is provided to an individual with hepatic encephalopathy is a polyethylene glycol (PEG) containing composition. PEG provides effective bowel cleansing in a once daily dosing, which removes an additional level of relative complexity for the cognitively impaired hepatic encephalopathy patient (as compared to multi-daily dosing of lactulose). As such, in embodiments of the compositions, methods, and kits described herein that include PEG, the necessary amount of laxative is delivered in a single dose using the food based delivery vehicle described herein.

Delivery of a Palatable Therapeutic Formulation

Typically, a traditional therapeutic regimen for the treatment and prevention of hepatic encephalopathy includes one or more unpalatable elements, such as, for example, lactulose. Patients with hepatic encephalopathy often have difficulty ingesting one or more unpalatable therapeutic agents thus leading to poor compliance with the therapeutic regiment.

Described herein are compositions, methods, and kits that provide a palatable formulation for delivering an unpalatable therapeutic agent. That is, in some embodiments of the compositions, methods, and kits described herein, one or more unpalatable therapeutic agents are mixed with one or more palatable food ingredients thus forming a palatable food item that contains the one or more unpalatable therapeutic agents. In this way, the formulation at least masks the unpalatable taste of the one or more unpalatable therapeutic agents, thus, promoting compliance by improving palatability.

Methods for Treating or Preventing Hepatic Encephalopathy

Described is a method for treating or preventing hepatic encephalopathy in an individual in need thereof. In some embodiments of the method, the method comprises providing to the individual a therapeutic composition comprising a delivery modality comprising one or more food ingredients, and one or more therapeutic agents, wherein the one or more therapeutic agents are mixed together with the one or more food ingredients. In some embodiments of the method, the laxative comprises polyethelyne glycol. In some embodiments of the method, the polyethelyne glycol is PEG 3350. In some embodiments of the method, the laxative comprises lactulose. In some embodiments of the method, the one or more therapeutic agents comprises an antibiotic. In some embodiments of the method, the antibiotic comprises rifaxamin. In some embodiments of the method, the antibiotic comprises neomycin. In some embodiments of the method, the antibiotic comprises metronidazole. In some embodiments of the method, the antibiotic comprises nitanoxinide. In some embodiments of the method, the one or more therapeutic agents comprises sodium benzoate. In some embodiments of the method, the one or more therapeutic agents comprises AST-120. In some embodiments of the method, the one or more therapeutic agents comprises OCR-002. In some embodiments of the method, the one or more therapeutic agents comprises L-ornithine-L-aspartate. In some embodiments of the method, the one or more therapeutic agents comprise sodium benzoate and one or more therapeutic agents comprise L-ornithine-L-aspartate. In some embodiments of the method, the one or more therapeutic agents comprise electrolytes. In some embodiments of the method, the therapy delivery modality comprises a food bar with the one or more therapeutic agents incorporated therein. In some embodiments of the method, the therapy delivery modality comprises a meal with the one or more therapeutic agents incorporated therein. In some embodiments of the method, the therapy delivery modality comprises a homogenous mixture of the one or more therapeutic agents and the one or more food ingredients. In some embodiments of the method, the one or more therapeutic agents mixed with the one or more food ingredients is heated. In some embodiments of the method, the one or more therapeutic agents mixed with the one or more food ingredients are heated by mixing the one or more therapeutic agents mixed with the one or more food ingredients with a heated binder. In some embodiments of the method, the binder comprises cocoa butter. In some embodiments of the method, the binder comprises coconut oil. In some embodiments of the method, the heated binder is heated to a temperature above 90 degrees Fahrenheit and the one or more therapeutic agents is mixed with the heated binder when it cools to a temperature below 80 degrees Fahrenheit. In some embodiments of the method, the one or more food ingredients are a USP-NF grade ingredient.

Compositions for Treating or Preventing Hepatic Encephalopathy

Described herein is a composition comprising a laxative; an antibiotic; and a food ingredient; wherein the composition comprises a mixture of the laxative the antibiotic and the food ingredient. In some embodiments of the composition, the laxative comprises polyethelyne glycol. In some embodiments of the composition, the polyethelyne glycol is PEG 3350. In some embodiments of the composition, the laxative comprises lactulose. In some embodiments of the composition, the antibiotic comprises rifaxamin. In some embodiments of the composition, the antibiotic comprises neomycin. In some embodiments of the composition, the antibiotic comprises metronidazole. In some embodiments of the composition, the antibiotic comprises nitanoxinide. In some embodiments of the composition, the composition further comprises sodium benzoate. In some embodiments of the composition, the composition further comprises AST-120. In some embodiments of the composition, the composition further comprises OCR-002. In some embodiments of the composition, the composition further comprises L-ornithine-L-aspartate. In some embodiments of the composition, the composition further comprises sodium benzoate and L-ornithine-L-aspartate. In some embodiments of the composition, the composition further comprises electrolytes. In some embodiments of the composition, the mixture comprises a food bar. In some embodiments of the composition, the composition further comprises a meal. In some embodiments of the composition, the mixture is a homogenous mixture. In some embodiments of the composition, the mixture is heated. In some embodiments of the composition, the mixture is heated by mixing it with a heated binder. In some embodiments of the composition, the binder comprises cocoa butter. In some embodiments of the composition, the binder comprises coconut oil. In some embodiments of the composition, the heated binder is heated to a temperature above 90 degrees Fahrenheit and the one or more therapeutic agents is mixed with the heated binder when it cools to a temperature below 80 degrees Fahrenheit. In some embodiments of the composition, the one or more food ingredients are a USP-NF grade ingredient.

Kits for Treating or Preventing Hepatic Encephalopathy

Described herein is a kit comprising one or more compositions comprising a laxative; an antibiotic; and a food ingredient; wherein the composition comprises a mixture of the laxative the antibiotic and the food ingredient. In some embodiments of the composition, the laxative comprises polyethelyne glycol. In some embodiments of the kit, the polyethelyne glycol is PEG 3350. In some embodiments of the kit, the laxative comprises lactulose. In some embodiments of the kit, the antibiotic comprises rifaxamin. In some embodiments of the kit, the antibiotic comprises neomycin. In some embodiments of the kit, the antibiotic comprises metronidazole. In some embodiments of the kit, the antibiotic comprises nitanoxinide. In some embodiments of the kit, the composition further comprises sodium benzoate. In some embodiments of the kit, the composition further comprises AST-120. In some embodiments of the kit, the composition further comprises OCR-002. In some embodiments of the kit, the composition further comprises L-ornithine-L-aspartate. In some embodiments of the kit, the composition further comprises sodium benzoate and L-ornithine-L-aspartate. In some embodiments of the kit, the composition further comprises electrolytes. In some embodiments of the kit, the mixture comprises a food bar. In some embodiments of the kit, the composition further comprises a meal. In some embodiments of the kit, the mixture is a homogenous mixture. In some embodiments of the kit, the mixture is heated. In some embodiments of the kit, the mixture is heated by mixing it with a heated binder. In some embodiments of the kits, the binder comprises cocoa butter. In some embodiments of the kits, the binder comprises coconut oil. In some embodiments of the kits, the heated binder is heated to a temperature above 90 degrees Fahrenheit and the one or more therapeutic agents is mixed with the heated binder when it cools to a temperature below 80 degrees Fahrenheit. In some embodiments of the kit, the one or more food ingredients is a USP-NF grade ingredient.

### DETAILED DESCRIPTION

Described herein are compositions, methods, and kits for providing therapy to an individual to treat and/or prevent hepatic encephalopathy. More specifically, described herein are compositions for treating or preventing hepatic encephalopathy that comprise delivery modalities and formulations for oral delivery of a therapeutic agent to an individual in need thereof.

A delivery modality as described herein comprises a food item or meal, which include numerous further embodiments. For example, a delivery modality may comprise a drink or a shake. For example, a delivery modality may comprise a candy or a gum.

In general, the delivery modalities described herein comprise an edible item, comprising, for example, a food, a drink, or a candy, and the delivery modalities further have one or more therapeutic agents incorporated therein.

In general, the compositions, methods, and kits for treating or preventing hepatic encephalopathy described herein comprise a delivery modality comprising one or more edible items and one or more therapeutic agents that are incorporated into the delivery modality.

In some embodiments of the compositions, methods, kits described herein a composition for treating or preventing hepatic encephalopathy comprises one or more therapeutic agents mixed with one or more food ingredients. In some embodiments of the compositions, methods, and kits described herein, the mixture is a homogenous mixture of the one or more therapeutic agents and the one or more food ingredients.

In an exemplary embodiment, one or more therapeutic agents for treating or preventing hepatic encephalopathy are mixed together with food ingredients to form a food bar. In this embodiment, the food bar comprises one or more of any ingredients typically found in a food bar such as, for example, chocolate and/or nuts, and/or fruit, and/or sweetener thus forming a delivery modality comprising a bar. A therapeutic agent is incorporated into the bar such that the bar serves as modality for delivering the therapeutic agent to an individual who eats the bar. The bar may be formed by, for example, mixing one or more food ingredients with the one or more therapeutic agents. In some embodiments of the compositions, methods, and kits described herein, one or more food ingredients are added to the bar as a coating. In some embodiments of the compositions, methods, and kits described herein, a mixture of the one or more therapeutic agents and the one or more food ingredients is heated to form the bar comprising the one or more food ingredients as well as the one or more therapeutic agents incorporated therein. In some embodiments of the compositions, methods, and kits described herein, the one or more therapeutic agents and the one or more food ingredients are combined together without mixing. In some embodiments of the compositions, methods, and kits described herein, the one or more therapeutic agents and the one or more food ingredients are combined without heating.

Mixing of one or more therapeutic agents and one or more food ingredients is used to generate essentially any conceivable food, beverage, candy, or gum. Heating of one or more of the elements of the mixture further provides coalescing as well as cooking of one or more of the food ingredients. For example, compositions for treating or preventing hepatic encephalopathy as described herein comprise meals such as, for example, soup, lasagna or chicken with rice. For example, the compositions for treating or preventing hepatic encephalopathy described herein comprise shakes and flavored drinks. The edible delivery modality is designed to be palatable to the individual and offer numerous different modalities that appeal to the varying tastes of different individuals in order to effectively deliver the one or more therapeutic agents incorporated therein.

The compositions for treating or preventing hepatic encephalopathy described herein comprise elements comprising one or more therapeutic agents and a delivery modality comprising one or more food ingredients. In some embodiments of the compositions, methods, and kits described herein, heating one or more elements of a composition for treating encephalopathy (i.e. the one or more therapeutic agents and/or the one or more food ingredients) is done at a temperature wherein the food ingredients tend to coalesce together while the therapeutic agents remain unchanged. In some embodiments of the compositions, methods, and kits described herein, neither the one or more food ingredients coalesce nor are the one or more therapeutic agents changed. In some embodiments of the compositions, methods, and kits described herein, the one or more food ingredients and the one or more therapeutic agents all coalesce together when heated. Typically, heat is not applied at or above a temperature at which a therapeutic property of one or more therapeutic agents of the composition are affected.

The effect of heating, as is well known, is determined by both the temperature and duration over which heat is applied. In some embodiments of the compositions, methods, and kits described herein, a composition for treating or preventing hepatic encephalopathy comprises a mixture of one or more therapeutic agents and one or more food ingredients which are heated to a temperature at or above 350 degrees Fahrenheit. In some embodiments of the compositions, methods, and kits described herein, a composition for treating or preventing hepatic encephalopathy comprises a mixture of one or more therapeutic agents and one or more food ingredients which are heated to a temperature at or above 300 degrees Fahrenheit. In some embodiments of the compositions, methods, and kits described herein, a composition for treating or preventing hepatic encephalopathy comprises a mixture of one or more therapeutic agents and one or more food ingredients which are heated to a temperature at or above 250 degrees Fahrenheit. In some embodiments of the compositions, methods, and kits described herein, a composition for treating or preventing hepatic encephalopathy comprises a mixture of one or more therapeutic agents and one or more food ingredients which are heated to a temperature at or above 200 degrees Fahrenheit. In some embodiments of the compositions, methods, and kits described herein, a composition for treating or preventing hepatic encephalopathy comprises a mixture of one or more therapeutic agents and one or more food ingredients which are heated to a temperature at or above 150 degrees Fahrenheit. In some embodiments of the compositions, methods, and kits described herein, a composition for treating or preventing hepatic encephalopathy comprises a mixture of one or more therapeutic agents and one or more food ingredients which are heated to a temperature at or above 100 degrees Fahrenheit. In some embodiments of the compositions, methods, and kits described herein, a composition for treating or preventing hepatic encephalopathy comprises a mixture of one or more therapeutic agents and one or more food ingredients which are heated to a temperature at or above 95 degrees Fahrenheit. In some embodiments of the compositions, methods, and kits described herein, a composition for treating or preventing hepatic encephalopathy comprises a mixture of one or more therapeutic agents and one or more food ingredients which are heated to a temperature at or above 90 degrees Fahrenheit. In some embodiments of the compositions, methods, and kits described herein, a composition for treating or preventing hepatic encephalopathy comprises a mixture of one or more therapeutic agents and one or more food ingredients which are heated to a temperature at or above 85 degrees Fahrenheit. In some embodiments of the compositions, methods, and kits described herein, a composition for treating or preventing hepatic encephalopathy comprises a mixture of one or more therapeutic agents and one or more food ingredients which are heated to a temperature at or above 80 degrees Fahrenheit. In some embodiments of the compositions, methods, and kits described herein, a composition for treating or preventing hepatic encephalopathy comprises a mixture of one or more therapeutic agents and one or more food ingredients which are heated to a temperature at or above 75 degrees Fahrenheit. In some embodiments of the compositions, methods, and kits described herein, the duration of heating is 1 hour or greater. In some embodiments of the compositions, methods, and kits described herein, the duration of heating is 45 minutes or greater. In some embodiments of the compositions, methods, and kits described herein, the duration of heating is 30 minutes or greater. In some embodiments of the compositions, methods, and kits described herein, the duration of heating is 20 minutes or greater. In some embodiments of the compositions, methods, and kits described herein, the duration of heating is 15 minutes or greater. In some embodiments of the compositions, methods, and kits described herein, the duration of heating is 10 minutes or greater. In some embodiments of the compositions, methods, and kits described herein, the duration of heating is 5 minutes or greater. In some embodiments of the compositions, methods, and kits described herein, the duration of heating is 1 minute or greater.

In some embodiments of the compositions, methods, and kits described herein, a composition for treating or preventing hepatic encephalopathy further comprises a binder, wherein a binder is an element used to bind one or more elements of the composition together. In some embodiments of the method, the binder is edible. In some embodiments of the method, the binder comprises cocoa butter. In some embodiments of the method, the binder comprises coconut oil.

In some embodiments of the compositions, methods, and kits described herein, a binder is heated and the elements of the composition are added to the heated binder. In some embodiments of the method, one or more elements of the composition are added to the binder at the hottest temperature to which it is heated. In some embodiments of the method, one or more elements of the composition are added to the heated binder after it has cooled. In some embodiments of the method, the binder is a liquid when heated so that when mixed with the elements of the composition, the elements coalesce or are “bound” together by the binder. In an exemplary embodiment, a binder in the composition is in a solid state at room temperature and a liquid state at a relatively small increase in temperature above room temperature. This exemplary binder is used in binding the elements of the composition when a solid composition is used such as, for example, a bar. In the liquid state the binder can coalesce the elements, and, as stated, the exemplary binder is in a liquid state at temperatures slightly above room temperature, thus the other elements of the composition are not heated due to the binder to a large extent which is especially important with respect to the therapeutic agents that are damaged at high temperatures. In some embodiments of the compositions, methods, and kits described herein, a binder is heated to a temperature at or above 95 degrees Fahrenheit and then one or more elements of the composition for treating or preventing hepatic encephalopathy are mixed together with it. In some embodiments of the compositions, methods, and kits described herein, a binder is heated to a temperature at or above 90 degrees Fahrenheit and then one or more elements of the composition for treating or preventing hepatic encephalopathy are mixed together with it. In some embodiments of the compositions, methods, and kits described herein, a binder is heated to a temperature at or above 85 degrees Fahrenheit and then one or more elements of the composition for treating or preventing hepatic encephalopathy are mixed together with it. In some embodiments of the compositions, methods, and kits described herein, a binder is heated to a temperature at or above 80 degrees Fahrenheit and then one or more elements of the composition for treating or preventing hepatic encephalopathy are mixed together with it. In some embodiments of the compositions, methods, and kits described herein, a binder is heated to a temperature at or above 75 degrees Fahrenheit and then one or more elements of the composition for treating or preventing hepatic encephalopathy are mixed together with it. In some embodiments of the compositions, methods, and kits described herein, a binder is heated to a temperature at or above 70 degrees Fahrenheit and then one or more elements of the composition for treating or preventing hepatic encephalopathy are mixed together with it. In some embodiments of the method, after being heated to an initial temperature, the binder is allowed to cool 30 degrees from the initial temperature before one or more elements of the composition for treating or preventing hepatic encephalopathy are added. In some embodiments of the method, after being heated to an initial temperature, the binder is allowed to cool 25 degrees from the initial temperature before one or more elements of the composition for treating or preventing hepatic encephalopathy are added. In some embodiments of the method, after being heated to an initial temperature, the binder is allowed to cool 20 degrees from the initial temperature before one or more elements of the composition for treating or preventing hepatic encephalopathy are added. In some embodiments of the method, after being heated to an initial temperature, the binder is allowed to cool 15 degrees from the initial temperature before one or more elements of the composition for treating or preventing hepatic encephalopathy are added. In some embodiments of the method, after being heated to an initial temperature, the binder is allowed to cool 10 degrees from the initial temperature before one or more elements of the composition for treating or preventing hepatic encephalopathy are added. In some embodiments of the method, after being heated to an initial temperature, the binder is allowed to cool 5 degrees from the initial temperature before one or more elements of the composition for treating or preventing hepatic encephalopathy are added. It is understood that numerous other edible binders are suited, non-limiting examples of which include lard, vegetable shortening, palm oil, butter, or margarine.

A number of therapeutic agents for treating or preventing hepatic encephalopathy are provided by different embodiments of the compositions methods, and kits described herein. In some embodiments of the compositions, methods, and kits described herein, the compositions for treating or preventing hepatic encephalopathy comprise a therapeutic agent comprising a laxative, wherein the composition further comprises a delivery modality in which the laxative is incorporated. In some embodiments of the method, the laxative comprises lactulose. In some embodiments of the method, the laxative comprises polyethelyne glycol. In some embodiments of the method, the type of polyethelyne glycol used is PEG 3350. In some embodiments of the compositions, methods, and kits described herein, the compositions for treating or preventing hepatic encephalopathy comprise a therapeutic agent comprising an antibiotic, wherein the composition further comprises a delivery modality in which the antibiotic is incorporated. In some embodiments of the method, the antibiotic comprises rifaxamin. In some embodiments of the method, the antibiotic comprises neomycin. In some embodiments of the method, the antibiotic comprises metronidazole. In some embodiments of the method, the antibiotic comprises nitazoxanide. An exemplary composition comprises a laxative and an antibiotic incorporated into the delivery modality, wherein said incorporation is achieved as described herein. As is understood, numerous laxatives and antibiotics are suitable for use in the compositions described herein.

In some embodiments of the compositions described herein, composition comprises 17 grams of PEG. Other doses of PEG are suitable for use with the compositions described herein including, for example, 16 grams of PEG, 15 grams of PEG, 14 grams of PEG, 13 grams of PEG, 12 grams of PEG, 11 grams of PEG, grams of PEG, 10 grams of PEG, 9 grams of PEG, 8 grams of PEG, 7 grams of PEG, 6 grams of PEG, 5 grams of PEG, 4 grams of PEG, 3 grams of PEG, 2 grams of PEG, and 1 gram of PEG.

Modular dosing of PEG is achieved using the compositions, methods, and kits described herein in some embodiments. For example, an individual prescribed 17 grams of PEG daily may be provided with, for example, a first composition comprising a food bar containing 10 grams of PEG and a second composition comprising a shake containing 7 grams of PEG. The individual of this example, may be, for example, provided the food bar containing 10 grams of PEG and the shake containing 7 grams of PEG in a kit. Likewise, an individual may be prescribed multiples of the same dose of PEG, for example, 17 grams of PEG to be taken three times daily. Such an individual, may be provided with, for example, a kit containing three meals—breakfast, lunch, and dinner—for example, that each have 17 grams of PEG incorporated therein. Providing the multiple doses to the individual in this manner has, for example, numerous benefits for an individual with diminished cognitive function as described herein.

In some embodiments of the compositions, methods, and kits described herein, a therapeutic agent in a composition for treating encephalopathy comprises AST-120. In some embodiments of the compositions, methods, and kits described herein, a therapeutic agent in a composition for treating encephalopathy comprises OCR-002. In some embodiments of the compositions, methods, and kits described herein, a therapeutic agent in a composition for treating encephalopathy comprises L-ornithine-L-aspartate (LOLA). In some embodiments of the compositions, methods, and kits described herein, a therapeutic agent in a composition for treating encephalopathy comprises sodium benzoate.

In some embodiments of the compositions, methods, and kits described herein a composition for treating or preventing hepatic encephalopathy further comprises an amount of electrolytes sufficient to replace electrolytes lost due to the actions of a laxative.

A delivery modality as described herein comprises an edible carrier of one or more therapeutic agents for treating hepatic encephalopathy. The delivery modality comprises one or more food ingredients. In some embodiments of the method, one or more food ingredients used comprise United States Pharmacopeial Convention (USP) grade ingredients. In some embodiments of the method, the entire delivery modality comprises USP food ingredients. A USP-NF grade ingredient or chemical comprises an ingredient or chemical that meets the standards as listed in monographs found in the United States Pharmacopeia and the National Formulary (USP-NF). A monograph includes the name of the ingredient or preparation, the definition, packaging, storage, and labeling requirements, and the specification. The specification consists of a series of tests, procedures for the tests, and acceptance criteria. Any and all USP-NF grade listed foods are suitable for use with the compositions, methods, and kits described herein.

In some embodiments of the compositions, methods, and kits described herein, one or more therapeutic agents for treating or preventing hepatic encephalopathy are mixed together with USP-NF grade food ingredients to form a food bar comprising one or more of any USP-NF grade food ingredients typically found in a food bar such as, for example, USP-NF grade chocolate and/or USP-NF grade nuts, and/or USP-NF grade fruit, and/or USP-NF grade sweetener thus forming a delivery modality comprising a bar.

In some embodiments of the compositions, methods, and kits described herein, a USP-NF grade food ingredient comprises a sweetener. In some embodiments of the compositions, methods, and kits described herein, a USP-NF grade sweetener comprises granulated sucrose. In some embodiments of the compositions, methods, and kits described herein a USP-NF grade sweetener comprises a syrup. In some embodiments of the compositions, methods, and kits described herein, a USP-NF grade sweetener comprises invert syrup. In some embodiments of the compositions, methods, and kits described herein, a USP-NF grade sweetener comprises erythritol. In some embodiments of the compositions, methods, and kits described herein, a USP-NF grade sweetener comprises maltodextrin. In some embodiments of the compositions, methods, and kits described herein, a USP-NF grade sweetener comprises dextrin.

In some embodiments of the compositions, methods, and kits described herein, a USP-NF grade ingredient comprises a texturizer. In some embodiments of the compositions, methods, and kits described herein, a USP food ingredient comprises cocoa butter. In some embodiments of the compositions, methods, and kits described herein, a USP-NF grade ingredient comprises malic acid. In some embodiments of the method, a USP-NF grade ingredient comprises citric acid. In some embodiments of the compositions, methods, and kits described herein, a USP-NF grade ingredient comprises lemon oil. In some embodiments of the compositions, methods, and kits described herein, a USP-NF grade ingredient comprises vanilla flavor.

In some embodiments of the compositions, methods, and kits described herein, the compositions described herein comprise one or more microencapsulated therapeutic agents. Microencapsulation comprises coating small particles of a gas, a liquid, a solid, or a combination thereof with a coating. The coating shields the gas, liquid, solid, or combination thereof so that, for example, when a microencapsulated gas, liquid, solid, or combination thereof is ingested by an individual, the coating prevents the microencapsulated gas, liquid, solid, or combination from contacting the taste buds of the individual. Thus, the individual does not taste or experience the texture of the microencapsulated gas, liquid, solid, or combination thereof when ingested.

Non-limiting examples of coatings suitable for use with the compositions, methods, and kits described herein include ethyl cellulose, polyvinyl alcohol, gelatin, sodium alginate, and chitin.

Microencapsulation comprises coating small particles of a gas, a liquid, a solid, or a combination thereof with a coating. The coating shields the gas, liquid, solid, or combination thereof so that, for example, when a microencapsulated gas, liquid, solid, or combination thereof is ingested by an individual, the coating prevents the microencapsulated gas, liquid, solid, or combination from contacting the taste buds of the individual. Thus, the individual does not taste or experience the texture of the microencapsulated gas, liquid, solid, or combination thereof when ingested.

Non-limiting examples of coatings suitable for use with the compositions, methods, and kits described herein include ethyl cellulose, polyvinyl alcohol, gelatin, sodium alginate, lipids, waxes, proteins (e.g. casein, gelatin albumin), cellulose and hemi-cellulose, starch, gums and polymers. and chitin.

In some embodiments of the compositions, methods, and kits described herein, a laxative provided in a bowel preparation regimen is microencapsulated. In some embodiments of the compositions, methods, and kits described herein, a laxative is dissolved in a liquid and a plurality of small quantities of the liquid are coated. In some embodiments of the compositions described herein, a plurality of small quantities of laxative are encapsulated together with small quantities of other active agents.

In some embodiments of the compositions, methods, and kits described herein, one or more food ingredients have a plurality of one or more microencapsulated therapeutic agents incorporated therein. That is, these compositions comprise a food item such as, for example, a food bar that has microencapsulated laxative (i.e. a plurality of microcapsules containing laxative) are incorporated therein. In some embodiments of the compositions, methods, and kits described herein, microencapsulated laxative is mixed together with one or more food ingredients to form a homogenous mixture. In some embodiments of the compositions, methods, and kits described herein, one or more food ingredients comprise United States Pharmacopeial Convention (USP) grade foods.

Microencapsulated therapeutic agents are released when the coatings surrounding the therapeutic agents are penetrated or broken down. In some embodiments of the compositions, methods, and kits described herein, coatings are selected to be broken down by digestive acids and enzymes that are normally found in the digestive tract of an individual. In some embodiments of the compositions, methods, and kits described herein, an individual who ingests a composition comprising microencapsulated therapeutic agents is provided an acidic drink to ingest immediately after ingestion of the composition so that the acid in the acidic drink will release the therapeutic agents from the coatings surrounding them.

The properties of the coatings determine the timing of release of the therapeutic agent contained therein in a predictable manner thus providing a mechanism for controlled release of a therapeutic agent. For example, in some embodiments, a composition for treating an individual with hepatic encephalopathy comprises a first therapeutic agent coated with a first coating and a second therapeutic agent coated with a second coating, wherein the first coating is known to release its contents at a different part of the intestinal tract than the second coating.

Described herein are kits in which one or more compositions for treating encephalopathy are provided. In some embodiments of the kits described herein, a kit comprises more than one composition embodiment. For example, in one embodiment of the kits described herein, a kit comprises a first composition for treating or preventing hepatic encephalopathy wherein the therapeutic agent is an antibiotic and a second composition for treating or preventing hepatic encephalopathy wherein the therapeutic agent is a laxative. Likewise, a kit may comprise a first composition for treating or preventing hepatic encephalopathy wherein the delivery modality comprises a bar and a second composition for treating or preventing hepatic encephalopathy wherein the delivery modality comprises a shake.

Described herein is a method for treating or preventing hepatic encephalopathy in an individual in need thereof. The method comprises providing a one or more compositions for treating encephalopathy or a kit as described herein to an individual in need. Treatment may be both therapeutic as well as preventative.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for treating or preventing hepatic encephalopathy in an individual in need thereof, comprising
providing to the individual a therapeutic composition comprising
a delivery modality comprising one or more food ingredients; and
one or more therapeutic agents;
wherein the one or more therapeutic agents are mixed together with the one or more food ingredients.

2. The method of claim 1, wherein the one or more therapeutic agents comprise a laxative.

3. The method of claim 2, wherein the laxative comprises polyethelyne glycol.

4. The method of claim 3, wherein the polyethelyne glycol is PEG 3350.

5. The method of claim 2, wherein the laxative comprises lactulose.

6. The method of claim 1, wherein the one or more therapeutic agents comprises an antibiotic.

7. The method of claim 6, wherein the antibiotic comprises rifaxamin.

8. The method of claim 6, wherein the antibiotic comprises neomycin.

9. The method of claim 6, wherein the antibiotic comprises metronidazole.

10. The method of claim 6, wherein the antibiotic comprises nitanoxinide.

11. The method of claim 1, wherein the one or more therapeutic agents comprises sodium benzoate.

12. The method of claim 1, wherein the one or more therapeutic agents comprises AST-120.

13. The method of claim 1, wherein the one or more therapeutic agents comprises OCR-002.

14. The method of claim 1, wherein the one or more therapeutic agents comprises L-ornithine-L-aspartate.

15. The method of claim 1, wherein the one or more therapeutic agents comprise sodium benzoate and L-ornithine-L-aspartate.

16. The method of claim 1, wherein the one or more therapeutic agents comprise electrolytes.

17. The method of claim 1, wherein the therapy delivery modality comprises a food bar with the one or more therapeutic agents incorporated therein.

18. The method of claim 1, wherein the therapy delivery modality comprises a meal with the one or more therapeutic agents incorporated therein.

19. The method of claim 1, wherein the therapy delivery modality comprises a beverage with the one or more therapeutic agents incorporated therein.

20. The method of claim 1, wherein the therapy delivery modality comprises a homogenous mixture of the one or more therapeutic agents and the one or more food ingredients.

21. The method of claim 1, wherein the one or more therapeutic agents mixed with the one or more food ingredients is heated.

22. The method of claim 21, wherein the one or more therapeutic agents mixed with the one or more food ingredients are heated by mixing the one or more therapeutic agents mixed with the one or more food ingredients with a heated binder.

23. The method of claim 22, wherein the binder comprises cocoa butter.

24. The method of claim 22, wherein the binder comprises coconut oil.

25. The method of claim 22, wherein the heated binder is heated to a temperature above 90 degrees Fahrenheit and the one or more therapeutic agents is mixed with the heated binder when it cools to a temperature below 80 degrees Fahrenheit.

26. The method of any one of claims 1-25, wherein the one or more food ingredients is a USP-NF grade ingredient.

27. A composition comprising
a laxative;
an antibiotic; and
a food ingredient;
wherein the composition comprises a mixture.

28. The composition of claim 26, wherein the laxative comprises polyethelyne glycol.

29. The composition of claim 27, wherein the polyethelyne glycol is PEG 3350.

30. The composition of claim 27, wherein the laxative comprises lactulose.

31. The composition of claim 30, wherein the antibiotic comprises rifaxamin.

32. The composition of claim 26, wherein the antibiotic comprises neomycin.

33. The composition of claim 26, wherein the antibiotic comprises metronidazole.

34. The composition of claim 26, wherein the antibiotic comprises nitanoxinide.

35. The composition of claim 26, comprising sodium benzoate.

36. The composition of claim 26, comprising AST-120.

37. The composition of claim 26, comprising OCR-002.

38. The composition of claim 26, comprising L-ornithine-L-aspartate.

39. The composition of claim 26, comprising sodium benzoate and L-ornithine-L-aspartate.

40. The composition of claim 26, comprising electrolytes.

41. The composition of claim 26, wherein the mixture comprises a food bar.

42. The composition of claim 26, wherein the mixture comprises a meal.

43. The composition of claim 26, wherein the mixture comprises a beverage.

44. The composition of claim 26, wherein the mixture is a homogenous mixture.

45. The composition of claim 26, wherein the mixture is heated.

46. The composition of claim 43, wherein the mixture is heated by mixing it with a heated binder.

47. The composition of claim 46, wherein the binder comprises cocoa butter.

48. The composition of claim 46, wherein the binder comprises coconut oil.

49. The composition of claim 46, wherein the heated binder is heated to a temperature above 90 degrees Fahrenheit and the one or more therapeutic agents is mixed with the heated binder when it cools to a temperature below 80 degrees Fahrenheit.

50. The composition of any one of claims 26-49, wherein the one or more food ingredients is a USP-NF grade ingredient.
